(19) United States
(12) Patent Application Publication
(10) Pub. No.: US 2025/0205464 A1
(43) Pub. Date: Jun. 26, 2025

(54) MULTIMODAL NEUROMODULATORY PAIN MANAGEMENT AND ACTIVE VECTORED TRANSDERMAL PHARMACEUTICAL PLATFORM DELIVERY SYSTEM

(71) Applicant: Vector Science & Therapeutics, Inc., Lodi, WI (US)

(72) Inventors: William Bradley Worthington, Nashville, TN (US); Barry Hix, Birmingham, AL (US); Thomas J. Bachinski, Lakeville, MN (US)

(21) Appl. No.: 19/063,568

(22) Filed: Feb. 26, 2025

Related U.S. Application Data
(63) Continuation-in-part of application No. 18/956,608, filed on Nov. 22, 2024.
(60) Provisional application No. 63/602,038, filed on Nov. 22, 2023.

(51) Int. Cl. A61M 37/00; A61K 41/00; A61M 19/00; A61N 1/36; A61N 2/00; A61N 2/02

(52) U.S. Cl. CPC A61M 37/00; A61K 41/0047; A61M 19/00; A61N 1/36021; A61N 2/002; A61N 2/008; A61N 2/02; A61M 2037/0007; A61M 2205/3303; A61M 2205/3317

(57) ABSTRACT

Provided is a multifunctional, multimodal, wearable platform device, including in the form of a patch, capable of delivering fluidic elements (including chemical compounds) through the skin to a subject in need thereof. The device is also capable of assisting in pain management via magnetic fields, electrical stimulation, kinetic energy, light energy, and other forms of non-chemical or non-pharmacological intervention.

### CROSS-REFERENCE TO RELATED APPLICATIONS

This application is a continuation-in-part and claims benefit of U.S. patent application Ser. No. 18/956,608 filed Nov. 22, 2024, which claims benefit of U.S. Provisional Application No. 63/602,038 filed Nov. 22, 2023, the specifications of which are incorporated herein in their entirety by reference.

### FIELD OF THE INVENTION

The present invention is a novel active transdermal drug delivery system incorporating and optimizing the synergistic projection of neuromodulatory transcutaneous electrotherapeutic and converse opposing electromagnetic fields to provide, project or vector, non-opioid, non-systemic multimodal pain control with or without the use of analgesic compounds, delivered/vectored and maintained at mammalian spatial targeted tissue using artificial intelligence and/or device-to-patient direct feedback-controlled application.

### BACKGROUND OF THE INVENTION

Chronic pain is a significant medical, social, and economic issue, as annual costs attributed to chronic pain alone in the United States range from $560 to $635 billion dollars which includes healthcare expenses, disability, and lost productivity.

Systemic analgesics, while useful for controlling pain, are associated with considerable side effects and toxicities. This is particularly true for systemic opioid analgesics, which even at recommend clinical doses may cause respiratory depression, and are responsible each year for at least tens of thousands of overdose deaths in the United States alone, and many more globally. Thus, there is a pressing need for effective alternatives to systemic analgesic treatment, especially systemic opioid analgesic treatment.

One such possibility lies in the use of wearable medical devices. Wearable medical devices may be used to deliver analgesic compounds or other compounds to a tissue of interest in need of such treatment. However, traditionally, such devices have lacked the ability to target specific tissues, particularly tissues along a specific vector and/or at a specific depth and have also lacked the ability to “maintain” such analgesics compounds or other compounds in said tissue in need of treatment so that they are able to carry out their mechanism of action and resultant analgesic effect, and/or other therapeutic non-analgesic effects, at a local site of action. There is thus a need for a wearable medical device capable of delivering therapeutic compounds, including analgesic compounds, to a site of action, including a site of action along a specific vector and/or at a specific spatial depth and therapeutic concentration, that is also capable of “maintaining” said compounds at said site of action.

### BRIEF SUMMARY OF THE INVENTION

It is an objective of the present invention to provide systems, devices, and methods that allow for transdermal drug delivery (e.g., active or passive transdermal delivery) incorporating and optimizing the synergistic, simultaneous, projection of neuromodulatory transcutaneous electrotherapeutic and magnetic fields to provide, project or vector therapeutic compounds (especially opioid-free analgesics) to a tissue in need of treatment, as specified in the independent claims. Embodiments of the invention are given in the dependent claims. Embodiments of the present invention can be freely combined with each other if they are not mutually exclusive.

In some embodiments, the present platform invention comprises a novel acute and chronic pain management platform incorporating a combination of transcutaneous, non-invasive, patient-controlled inventions, which include active transdermal pharmacological and non-pharmacological (medical device, drug delivery) interventions. Pharmacological interventions may include active pharmaceutical ingredients, such as, nonsteroidal anti-inflammatory drugs (NSAIDs), non-opioid analgesic drugs and compounds, antidepressants, anti-inflammatory and anticonvulsant therapeutics, as well as topical or infiltrated sodium channel blockers, NMDA receptor antagonists, TRPV agonists, steroids, and central alpha two agonist therapeutics.

Nonpharmacologic combined and included interventions may also include cognitive behavioral feedback therapies, photobiologic, neuromodulatory magnetic and/or transcutaneous neurostimulation techniques, acupuncture, acupressure, and other complementary and alternative therapeutic techniques.

In some embodiments, the present invention utilizes a single mode of action to treat pain, or maintain homoeostasis, in a subject (e.g., a mammal).

In some embodiments, the present invention utilizes two independent but synergistic modes of action to treat pain, or maintain homeostasis in a subject (e.g., a mammal).

In some embodiments, the present invention combines three or more synergistic modes of action to treat pain, or maintain homeostasis in a subject (e.g., a mammal).

In some embodiments, the present invention features a dual electrode ring. In some embodiments, the dual electrode ring is capable of delivering TENS, constant direct current, iontophoretic force, magnetophoretic or other appropriate forms of stimulation disclosed herein.

One of the unique and inventive technical features of the present invention is the ability to implement patient-empowering “point and shoot” analgesia, alone or in combination with vectored active transdermal, anti-inflammatory treatment, local anesthetic treatment, and/or multimodal, preemptive, and/or preventative analgesic/anti-inflammatory therapy applied to a specific targeted tissue, as a single treatment or as part of continuous treatment, via a wearable therapy device. Another unique and inventive technical feature of the present invention is the combination of more than one of the following treatment modalities, including novel multimodal non-invasive opioid-free magnetic converse opposing electromagnetic vectoring fields, transcutaneous electrical stimulation and active transdermal delivery of one or more chemical compounds, including but not limited to active pharmaceutical ingredients. Without wishing to limit the invention to any theory or mechanism, it is believed that the technical features of the present invention advantageously provide and/or apportions for patient empowering multimodal opioid free point of care non-invasive analgesia and anesthesia, and furthermore provides for non-invasive anesthetization of tissue prior to transdermal delivery of chemical compound to a patient in need thereof. None of the presently known prior references or works have the unique inventive technical features of the present invention.

Without wishing to limit the invention to any theory or mechanism, it is believed that magnetic converse opposing electromagnetic vectoring fields are advantageous in that they require no power source and are constitutively active. Without wishing to limit the invention to any theory or mechanism, it is believed that different combinations/permutations of different transcutaneous electrical nerve stimulation (TENS) frequencies, alternating current and direct current, pulse width and the like, produce, vector and project differing forms, cycles, and the like, of electromotive force that will affect polar substances in varying ways. Without wishing to limit the invention to any theory or mechanism, it is believed that vectored magnetic fields tend to push compounds laterally relative to the electrode, whereas vectored electric force from TENS pushes perpendicularly relative to the electrode, and that both significantly affect transdermal transit time and depth, enabling i) pushing compounds to a given target depth and ii) subsequently “pulling back” such that the compound stays or “spatially dwells” at a given target site at a maintained therapeutic concentration.

In some embodiments, magnetic fields may be produced biomechanically at micro scale within tissue, along the axis of electrical potential. In some embodiments, electrical signals may be harvested from the electric and magnetic fields projected by the electromagnetic field generator, and subsequently converted into magnetic field gradients. In some embodiments, the electrode may incorporate light emitting diodes of various light frequencies.

Moreover, the prior references teach away from the present invention. For example, standard transcutaneous electrical stimulation with increasing amplitude or pulse width causes local discomfort or pain near to the target electrode placement and may also cause painful muscle stimulation and spasm that is avoided using the present invention. The present invention provides for vectoring and active transdermal delivery of a variety of analgesic compounds, using enhanced, increased magnitudinal, transcutaneous and electromagnetic forces without causing pain or discomfort associated with the increased vectoring stimulus. A variety of therapeutic compounds are procurable vector candidates including sodium channel blockers, non-steroidal anti-inflammatory drugs, and NMDA receptor antagonists, and others obvious to one skilled in the art, vectored to targeted tissue as a single delivery or a continuous delivery.

Furthermore, the inventive technical features of the present invention contributed to a surprising result. For example, as taught by US 2009/0093669 A1 to Farone et al., a magnetic or electrical sphere of influence on a polar compound enhances transdermal drug movement greater than as caused by concentration gradient diffusion alone. Similarly, the combined vectored electrical and magnetic fields of influence applied to the epidermis by the present invention enhances transdermal delivery to the epidermis as compared to concentration gradient diffusion alone. The present invention, establishing and attaining greater non-painful vector force magnitude can similarly enhance, increase active vectored transdermal delivery, and maintained spatial therapeutic concentration for various drugs, drug classes, pro-drugs and therapeutic compounds, by enhancing both lateral and perpendicular transdermal delivery to the influenced tissue target of interest. In addition, and as previously not taught in the prior art, the present invention provides more effective and efficacious active transdermal delivery to a targeted tissue of interest by vectoring the active transdermal delivery of therapeutic compounds and maintaining the therapeutic compound at the targeted tissue site. The present invention provides for vectoring of therapeutic compounds, and maintaining compounds, with increased spatial dwell time on target and maintained spatial therapeutic efficacy by changing the electromotive force, alternating the electromotive force phase to maintain “time on station” at the desired site of pharmacodynamic effect. For example, the present invention may allow therapeutic vectoring and/or maintenance of spatial dwell time of a therapeutic compound to a precise, spatial tissue target, which may be a tissue fascial plane deep to the epidermis, the tissue fascial plane containing a peripheral sensory nerve targeted by an analgesic, anti-inflammatory, or anesthetic, said peripheral sensory nerve innervating a dermatome of interest to provide analgesia, anti-inflammation, or anesthesia as a single therapy or continuous therapy.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from consideration of the following detailed description presented in connection with the accompanying drawings in which:

FIG. 1 shows an exploded view of one embodiment of the present invention. In this embodiment, the device is a wearable, positional, placeable, platform or unit, placeable onto the skin, adjacent, near, or on top of, (for example) an injury, where said device may exert its functionality. For example, the converse opposing electromagnetic vectoring fields produced by the device may be used to influence and inhibit pain messaging mechanisms of the mammalian somatosensory system of a wearer of the present invention. The protective “bumper” allows for the wearer of the device to be ambulatory and provides a protective barrier between at least a portion of the device and the wearer. The platform reservoir is used to hold and disperse the desired compound or drug to and through the skin vectored to the targeted tissue. This mechanism of action is either accomplished by active and/or passive transdermal delivery. Another mechanism of action of the platform device pictured is, inter alia, the one or more conductive electrodes/hydrogels, sometimes deployed in pairs, that can be energized to provide electrostimulation that enhances drug delivery, manages pain, reduces edema, inhibits inflammation, and promotes tissue healing. The controlling unit is used to provide featured active functions to the design.

FIG. 2 shows another embodiment of the present platform invention. In this embodiment, a dual-electrode ring is present, which can deliver more complex electrical stimuli, enhancing the device's pain management functionality and vector active transdermal drug delivery. Additionally provided in this embodiment is an LED light ring, which can emit a variety of LED light wavelengths, with a variety of functions including, but not limited to: anti-bacterial functions, anti-inflammatory functions, analgesic functions, edema management, vasodilation, and other photobiologic therapies.

FIG. 3 shows another embodiment of the present platform invention. Shown in this embodiment is a self-contained power management system built into and/or mounted onto, the patch electrode of the present invention.

FIG. 4 shows one embodiment of the user controller of the present invention.

FIG. 5 shows one embodiment of the active energy electrode/patch/hydrogel reservoir of the present invention.

### DETAILED DESCRIPTION OF THE INVENTION

Following is a list of elements corresponding to a particular element referred to herein:
001 Wearable, multimodal device
100 Overlapping seal
101 Overlapping clear seal
110 Secondary seal ring
120 Primary seal ring
200 Electromagnetic Field Generator
201 Rotating electromagnet
210 Dual electrode ring
211 Light emitting diode
220 Active energy electrode
230 Electrode matrix
300 Bearing ring
400 Protective bumper
500 Reservoir
501 Port
510 Drug matrix
600 Power source
601 Lead wire
602 Sensor lead
603 Electrical connector
710 Printed circuit board control unit with integrated power source
800 User controller
801 User controller adjustment controls
810 User controller status screen

As used herein, a “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being. “Treatment,” as used herein, covers any administration or application of a therapeutic for disease in a mammal, including a human, and includes inhibiting the disease, arresting its development, or relieving the disease, for example, by causing regression, or restoring or repairing a lost, missing, or defective function; or stimulating an inefficient process.

As used herein, “clinical improvement” may refer to a noticeable reduction in the symptoms of a disorder, or cessation thereof.

As used herein, the terms “synergy” and “exponential synergy” may be used interchangeably. The term “synergy” may mean, inter alia, “produce a combined effect greater than the sum of their separate effects.” The term “exponential” may mean, inter alia, “becoming more and more rapid,” and/or according to the mathematical concept of exponential growth.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The terms “regress,” “regressing,” and “regression” may refer to a decrease in the size of a tumor or in the extent of cancer in the body. In some embodiments, “regression” may refer to a decrease in severity of the disease and/or decrease in the size of a tumor. In some embodiments, regression may generally refer to diminished symptoms without the disease completely disappearing. In certain cases, the beneficial effects that a subject derives from a prophylactic or therapeutic agent do not result in a cure of the disease or disorder. In some embodiments, symptoms of the disease may return.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower, but many times additive or synergistic, dosages than appropriate for their administration alone. Thus, multimodal co-administration is especially desirable in embodiments where the co-administration of the agents or therapies lowers the requisite dosage of a potentially harmful (e.g., toxic) agent(s), and/or when co-administration of two or more agents results in synergy of the beneficial effects of the therapies and decreasing dose dependent toxicities.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; genotype, pharmacogenetics, the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses or pulsed doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days, weekly, twice weekly, etc. Guidance can be found in the literature for appropriate dosages for given classes of pharmaceutical products. The innovation presented allows for the platform device to monitor a therapeutic's efficacy by real time monitoring of the therapeutic's effect and real time therapeutic adjustments. The multimodal platform allows for addition of additional therapeutics to optimize measured or reported outcome(s).

The exact amount, or dose, of the compositions required for efficacy will vary from subject to subject, depending on the genotype, species, age, weight, and general condition of the subject, the severity of the disorder being treated, the particular composition used, its mode of administration, enhanced delivery to targeted tissue, and the like. Thus, it is not possible to specify an exact amount for every composition. However, an appropriate amount can be determined by one of ordinary skill in the art using only routine experimentation, monitoring therapeutic effect, given the teachings herein.

The term “effective amount” as used herein refers to the amount of a therapy which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of any compound described herein is an amount sufficient to provide a therapeutic benefit in the treatment or management of a disease state, or to delay or minimize one or more symptoms associated with the presence of the disease state. A therapeutically effective amount of a compound described herein means an amount of therapeutic agent, alone or in combination with other therapies, which provides a therapeutic benefit in the treatment or management of the disease state. The term “therapeutically effective amount” can encompass an amount that improves overall therapy, reduces or avoids symptoms or causes of the disease state, or enhances the therapeutic efficacy of another therapeutic agent.

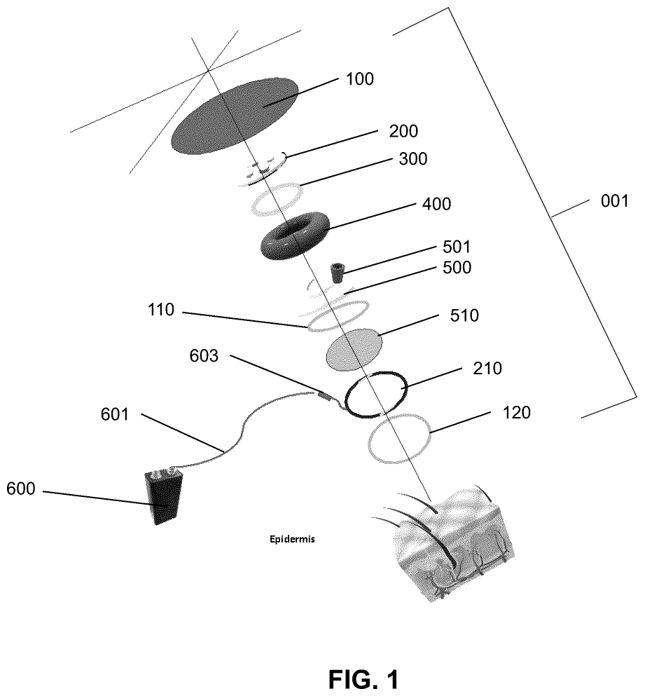

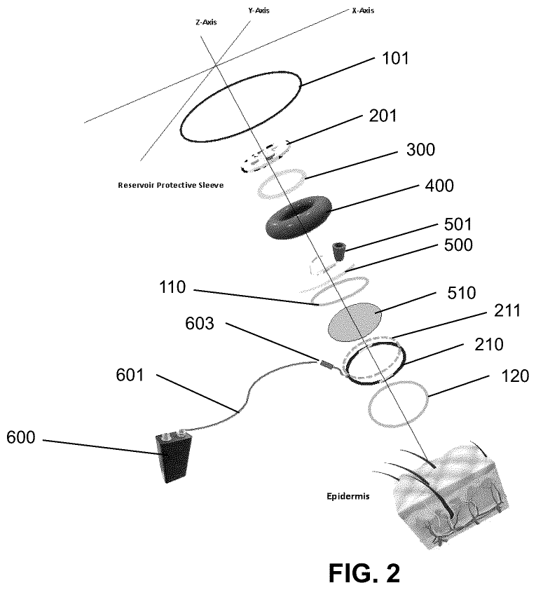

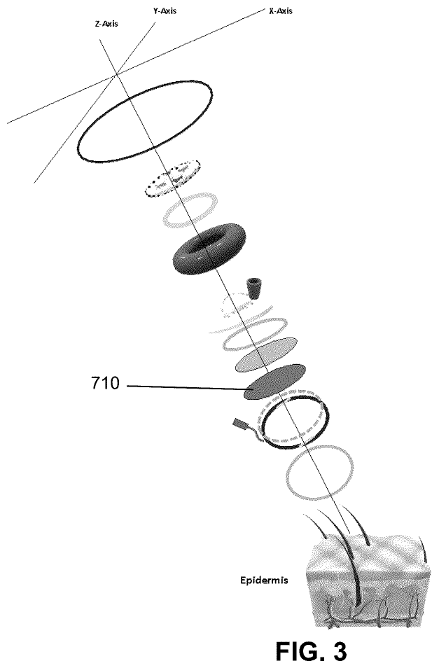

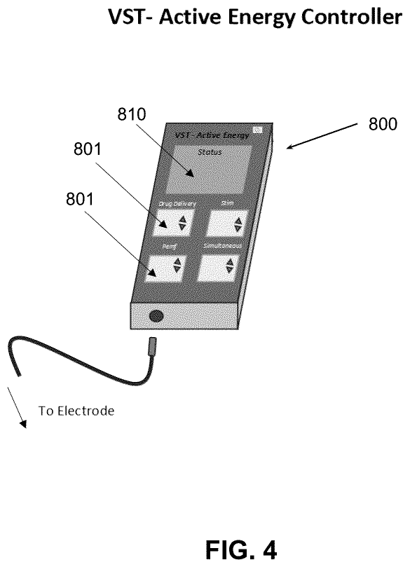

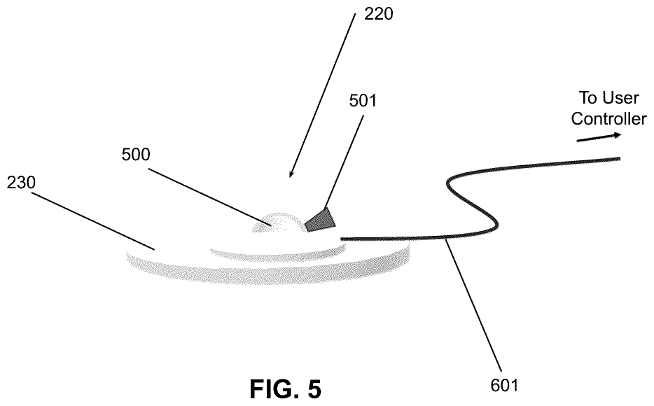

Referring now to FIGS. 1-5, in some embodiments, the present invention features a wearable multimodal device (001) comprising: a skin-contacting component; a therapeutic reservoir (500) containing at least one chemical compound therein, wherein the therapeutic reservoir (500) is fluidically coupled to the skin-contacting component at least one electromagnetic field generator (200) capable of generating and projecting electric and magnetic fields; a power source (600) electrically connected to the at least one electromagnetic field generator (200); a control unit electrically or wirelessly connected to the power source (600) and functionally connected to the therapeutic reservoir (500); wherein the control unit is configured to control the delivery of electrical power from the power source (600) to the electromagnetic field generator (200) thereby generating electric and magnetic fields; wherein the control unit is configured to control the delivery of the at least one chemical compound from the therapeutic reservoir (500) to the skin-contacting component; wherein the electric and magnetic fields generated and projected from the electromagnetic field generator are projected at least partially into an epidermis of a subject (e.g., a mammal) in need of therapy or analgesia, the skin-contacting component delivers the chemical compound to at least the epidermis of the subject (e.g., a mammal) in need of therapy or analgesia, and wherein the electric and magnetic fields generated and projected from the electromagnetic field generator at least partially hold the at least one chemical compound in, on, or at a treatment site for a dwell time, wherein the treatment site comprises a site where the at least one chemical compound exerts at least one mechanism of action.

In some embodiments, the electric and magnetic fields generated and projected from the electromagnetic field generator at least partially hold the at least one chemical compound in, on, or at a treatment site for a dwell time, wherein the treatment site comprises a site where the at least one chemical compound exerts at least one mechanism of action.

In some embodiments, the treatment site comprises a site of action at which the chemical compound exerts its mechanism of action, pharmacodynamic effect, or other effect on some portion of the body of the subject (e.g., mammal) in need of therapy or analgesia. In some embodiments, the treatment site is the epidermis. In some embodiments, the treatment site is the dermis, epidermis, hypodermis or other targeted sites for therapy to a subject (e.g., mammal) in need thereof. In some embodiments, the treatment site is deep to the skin, and may comprise any connective tissue, epithelial tissue, muscle tissue, and nervous tissue of the subject in need of therapy of analgesia. In some embodiments, the treatment site may be any cells, tissues, organs, and/or organ systems of the subject in need of therapy or analgesia. In some embodiments, the treatment site may be any part of the respiratory system, digestive system, gastrointestinal system, nervous system, endocrine system, cardiovascular system, circulatory system, reproductive system, urinary system, integumentary system, skeletal system, musculoskeletal system, immune system, lymphatic system, and the like, or any other tissue or organ of the body of the subject (e.g., mammal) in need of therapy or analgesia.

In some embodiments, the dwell time is definite and determined by the wearable multimodal device. In some embodiments, the dwell time may be selected by altering certain user-adjustable parameters of the wearable multimodal device. In some embodiments, the dwell time may be adjusted by the wearable multimodal device automatically. In some embodiments, the dwell time may be adjusted automatically, as a function, a response, to at least one feedback signal detected by at least one sensor and received by the control unit. In some embodiments, the dwell time is determined by at least one of the pharmacokinetic or pharmacodynamic properties of the at least one chemical compound. In some embodiments, the dwell time is anywhere from one second to one minute. In some embodiments, the dwell time is anywhere from one minute to one hour. In some embodiments, the dwell time is anywhere from one hour to two hours. In some embodiments, the dwell time is anywhere from one minute to one hour. In some embodiments, the dwell time is anywhere from two hours to four hours. In some embodiments, the dwell time is anywhere from four hours to six hours. In some embodiments, the dwell time is anywhere from six hours to eight hours. In some embodiments, the dwell time is anywhere from eight hours to 10 hours. In some embodiments, the dwell time is anywhere from 10 hours to 12 hours. In some embodiments, the dwell time is anywhere from 12 hours to 16 hours. In some embodiments, the dwell time is anywhere from 16 hours to 20 hours. In some embodiments, the dwell time is anywhere from 20 hours to 24 hours. In some embodiments, the dwell time is greater than 24 hours but less than 48 hours. In some embodiments, the dwell time is greater than 48 hours. In some embodiments, the dwell time is indefinite. In some embodiments, dwell time may be affected or adjusted by modulation of at least one of the following parameters of the converse opposing vectoring ferromagnetic or electromagnetic field or transdermal electromagnetic pulse: frequency, width, depth, waveform, rate, or amplitude of stimulation. In some embodiments, dwell time may be affected or adjusted by modulation of at least one of the following characteristics of the chemical compound delivered by the device: inertial characteristics, penetration, velocity, depth, directional vector, dipole moment, charge density, polarity, or size, at a treatment site. In some embodiments, the dwell time is affected by modulation of ionoto-magnetic projected, vectored, or electromagnetic active transdermal delivery signals, perpendicular and lateral to a target tissue.

In some embodiments, the dwell time may be affected or adjusted by specific variation or cycling of current density, and/or by reversing the actions performed by the multimodal, wearable device that caused “forward” movement or penetration into the skin, and/or by performing actions opposite to those initially performed that caused “forward” movement or penetration into the skin. In some embodiments, these “forward” and “reverse” actions may be cycled, i.e., first performing a “forward” cycle, then performing a “reverse” cycle, then subsequently performing a “forward” cycle, then subsequently performing another “reverse” cycle, and so on.

In some embodiments, the electric and magnetic fields generated and projected from the electromagnetic field generator at least partially hold the at least one chemical compound in, on, or at a treatment site for a dwell time via specific variation or cycling of current density, and/or via by reversing the actions performed by the multimodal, wearable device that caused “forward” movement or penetration into the skin, and/or by performing actions opposite to those initially performed that caused “forward” movement or penetration into the skin. In some embodiments, these “forward” and “reverse” actions may be cycled, i.e., first performing a “forward” cycle, then performing a “reverse” cycle, then subsequently performing a “forward” cycle, then subsequently performing another “reverse” cycle, and so on. In some embodiments, the present invention uses magnetic fields to interrupt pain signaling, including transmission and/or transduction of A-delta and C nerve fiber pain signals. This allows use of higher currents (in terms of amperage) than would otherwise be possible or tolerable, in order to move larger drug chemical compounds (e.g., drug molecules) more quickly and deeper into the tissue. By calculating or otherwise determining the electromotive force required to push the chemical compound (e.g., drug molecule) at a certain rate, the chemical compound's arrival at the targeted tissue can be determined. Once the chemical compound is “on site” at a treatment site (i.e., at least partially present in, on, or at the treatment site), the polarity of the electromotive force can be reversed and switched (i.e., oscillated), thus “pulling” and “pushing,” the chemical compound. In some embodiments, pulling and pushing is performed in alternating fashion, thereby keeping the chemical compound at least partially “on site” at the treatment site. This method for targeting (e.g., specifically vectoring to a treatment site) the chemical compound and maintaining its position at the treatment site (e.g., the tissue where the chemical compound exerts its desired mechanism of action) is more efficient than prior approaches. For example, because the movement of the chemical compound can be managed in this way, artificial intelligence can be incorporated for intelligent drug delivery based upon readings (e.g., by sensors). In some embodiments, this includes the use of noninvasive sensors (e.g., LEDs). In some embodiments, the sensors detect advanced glycation end products (AGEs). AGEs may be the product of fat and protein molecules being oxidized by sugar molecules. In some embodiments, when the sensors of the present device detect AGEs, the sensors transmit one or more signals to the control unit. In some embodiments, the control unit responds to this signal by implementing the release of insulin by the wearable, multimodal device of the present invention. In this example, this closed-loop system of detecting a condition (e.g., a physical or physiological condition) of the patient's body is signaled to the present invention's control unit, and then followed by subsequent delivery of chemical compound to the patient in response to this signal. This may include delivery of chemical compounds at a specific dose and/or concentration. This may be accomplished through the use of algorithms, artificial intelligence, or the like. As the system of the present invention “sees” more of these repetitive, closed-loop cycles, it can become more “intelligent” in determining the best-personalized drug delivery methodology, including for an individual patient. The present invention thereby provides for painlessly delivering targeted drugs to a specific location, and then holding those drugs in position at their site of desired action. The present invention features the advantages of allowing for non-systemic, painless, personalized, accurate, non-addictive, and intelligent drug delivery through the skin of the patient.

In some embodiments, the treatment site comprises a site where the at least one chemical compound exerts at least one mechanism of action.

In some embodiments, the dwell time may be affected by specific variation or cycling of current density. In some embodiments, the treatment site is at least one of the epidermis, dermis, hypodermis or deeper subcutaneous, fascial, or neural tissue targets of a subject in need thereof. In some embodiments, the chemical compound is delivered by the device via active transdermal delivery and/or passive transdermal delivery. “Active transdermal delivery” may be defined as the use of external stimuli, including external energy, to enhance the skin's permeability to drugs, which may involve the use of external energy to drive drugs across the skin, or may involve physical disruption of the stratum corneum or other skin components in order to facilitate drug transport across the skin. “Passive transdermal delivery” may be defined as methods of delivery drugs across the skin that rely primarily on passive diffusion of a drug across the skin, and include influencing of drug and vehicle interactions and optimization of formulation, in order to modify the stratum corneum structure.

In some embodiments, the wearable, multimodal device (001) of the present invention further comprises an overlapping seal (100) or a clear overlapping seal (101). In some embodiments, the overlapping seal (100) or the clear overlapping seal (101) seals the wearable, multimodal device (001) to the skin of the subject (e.g., a mammal) in need of therapy or analgesia, and/or protects the wearable, multimodal device (001) from water, dirt, and the like.

In some embodiments, the wearable, multimodal device (001) of the present invention further comprises a secondary seal ring (110). In some embodiments, the secondary seal sing (110) prevents leakage from the reservoir (500). In some embodiments, the present invention further comprises a primary seal ring (120). In some embodiment, the primary seal ring (120) prevents leakage from the reservoir (500).

In some embodiments, the wearable, multimodal device (001) of the present invention further comprises an electromagnetic field generator (200). In some embodiments, the electromagnetic field generator is capable of generating and projecting electric and magnetic fields. In some embodiments, the wearable, multimodal device (001) of the present invention further comprises a rotating electromagnetic (201). In some embodiments, the rotating electromagnetic (201) is capable of generating and projecting electric and magnetic fields. In some embodiments, the wearable, multimodal device (001) of the present invention further comprises a dual electrode ring (210). In some embodiments, the dual electrode ring (210) is capable of generating and projecting electric and magnetic fields.

In some embodiments, the wearable, multimodal device (001) of the present invention further comprises a light emitting diode (LED) (211). In some embodiments, the light emitting diode (211) is used to perform photobiomodulation, or deliver light therapy to a subject (e.g., a mammal) in need thereof, including but not limited to low-level light therapy (LLLT), and/or multispectral light therapy.

In some embodiments, the wearable, multimodal device (001) of the present invention further comprises an active energy electrode (220). In some embodiments, the active energy electrode (220) is capable of generating and projecting electric and magnetic fields. In some embodiment, the wearable, multimodal device (001) of the present invention further comprises an electrode matrix (230). In some embodiments, the electrode matrix (230) comprises electrodes (e.g., a plurality of electrodes). In some embodiments, the electrode matrix (230) is capable of generating and projecting electric and magnetic fields.

In some embodiments, the wearable, multimodal device (001) of the present invention further comprises a bearing ring (300). In some embodiments, the wearable, multimodal device (001) of the present invention further comprises a protective bumper (400).

In some embodiments, the wearable, multimodal device (001) of the present invention further comprises a reservoir (500). In some embodiments, the wearable, multimodal device (001) of the present invention further comprises one or more reservoirs (500). In some embodiments, the reservoir (500) contains at least one chemical compound. In some embodiments, the chemical compound is an active pharmaceutical ingredient (API). In some embodiments, the reservoir (500) is fluidically connected to the port (501). In some embodiments, the port dispenses chemical compounds for administration a subject (e.g., mammal) in need thereof. In some embodiments, the port (501) is fluidically connected to the drug matrix (510). In some embodiments, the drug matrix contains chemical compounds for administration to a subject (e.g., mammal) in need thereof.

In some embodiments, the wearable, multimodal device (001) of the present invention features a power source (600). In some embodiments, the power source is electrically or electronically connected to other components of the wearable, multimodal device (001) via the lead wire (601). In some embodiments, present invention further comprises a sensor, the sensor electrically or electronically connected to a sensor lead (602). In some embodiments, the sensor lead is electrically or electronically connected to the control unit. In some embodiments, the wearable, multimodal device (001) of the present invention further comprises an electrical connector (603). In some embodiments, the electrical connector connects electrical or electronic components of the wearable, multimodal device (001), including in some embodiments the power source (600) to the control unit.

In some embodiments, the wearable, multimodal device (001) of the present invention further comprises a printed circuit board control unit with integrated power source (710). In some embodiments, the control unit is a printed circuit board control unit with integrated power source (710). In some embodiments, the wearable, multimodal device (001) of the present invention further comprises a user controller (800). In some embodiments, the user controller (800) is electrically or electronically connected to electrical or electronic components of the wearable, multimodal device (001), including in some embodiments, the power source (600), the control unit, the printed circuit board control unit with integrated power source (710), the electromagnetic field generator (200), the rotating electromagnet (201), the dual electrode ring (210), the light emitting diode (211), the active energy electrode (220), the electrode matrix (230), the lead wire (601), the sensor lead (602), the electrical connector (603), the user controller adjustment controls (801), the user controller status screen (810), and the like.

In some embodiments, the present platform invention relates to a unimodal or multimodal wearable device, alone/independently, or in combination, to relieve pain and/or actively deliver transdermal compounds to a subject in need of analgesia or in need of other compounds to improve health status.

In some embodiments, the present platform invention relates to devices for the treatment of acute, chronic, and cancer pain, especially in an opioid-free modality. In some embodiments, the present invention comprises wearable devices and device platforms for delivery of compounds (e.g., drug molecules) through the skin. In some embodiments, an advantage of the present invention is an ability to deliver compounds through various types of skin. In some embodiments, an advantage of the present invention is an ability to vector compounds with a high molecular weight via active transdermal delivery.

In some embodiments, the present invention is modular in its design and function, and is unimodal or multimodal. In some embodiments, the present invention is a multimodal platform device, modular in its design and function. In some embodiments, the device is designed to be a platform technology that is capable of both treatment and diagnostic functions in pain management, population health, and transdermal drug delivery.

In some embodiments, the present invention's primary purpose is to manage pain, deliver, vector, compounds or other therapeutics, and provide diagnostic information to adjust and enhance current or future treatments. The present invention is used either singularly, unimodal, in combination, or multimodal, with one or more of the following technologies synchronized to allow for synergistic multimodal treatments. These technologies include, but are not limited to, ionic active or passive transdermal drug delivery, kinetic transdermal drug delivery, photonic transdermal drug delivery, passive absorption, and magnetophoretic and iontophoretic actions. Further, as related to pain management, the present invention can be programmed and/or used in combination, and/or programmed and/or used individually, or synergistically, along with, without limitation, one or more of the following treatment modalities: transcutaneous electrical nerve stimulation (TENS), interferential waveforms, high-frequency electrical stimulation, ultra-high frequency electrical stimulation, and/microcurrent stimulation. In some embodiments, the present invention features magnetic converse opposing electromagnetic vectoring fields and/or other electromagnetic fields, transcutaneous electrical nerve stimulation (TENS), and active transdermal drug delivery, or some combination thereof. In some embodiments, alternating magnetic converse opposing electromagnetic vectoring dynamic fields may provide for the attenuation or blocking of signal transduction and transmission of nociceptive nerve fibers, including A-delta fibers and C-fibers. In some embodiments, magnetic converse opposing electromagnetic vectoring alternating dynamic rotational magnetic fields may provide for the attenuation or blocking of signal transduction and transmission of nociceptive nerve fibers, including A-delta fibers and C-fibers. The present invention also has the functionality, ability, utility, to assist in, among other modalities, edema reduction, bone healing, and improving vascular tone and perfusion.

The present invention features a previously unrevealed combination of a wearable medical device platform, functioning independently, unimodally, and/or multimodally, projecting and/or vectoring direct current, electromagnetic, photobiologic, and/or dynamic rotational magnetic converse opposing electromagnetic neuromodulatory nociceptive inhibitory action, and/or features active transdermal delivery of therapeutic entities or compounds, including but not limited to, anti-nociceptive, anti-inflammatory, allopathic and/or homeopathic analgesic compounds or other active pharmaceutical ingredients, for example, insulin.

In some embodiments, the present invention comprises a device platform capable of projecting multimodal therapeutic actions at a distance from the targeted tissue by vectoring electromagnetic, photobiologic, and therapeutic compounds in a single delivered dosage or duty cycle, and/or a continuous delivery mode, or a multimodal duty cycle where different synergistic therapies can be delivered in a time-duration, and/or dose-contingent manner, allowing therapeutic integration of the desired effects.

In some embodiments, the present invention comprises a wearable platform device that may receive biological therapeutic feedback from integrated sensors that may provide guidance to optimize selected therapy, optimize combined therapies, and/or utilize artificial intelligence, to improve real-time simultaneous, or future Al-determined modulatory adjustments or transitions, to improve duty cycle efficacy and enhance personal or population health and safety. Importantly, the present invention is capable of providing non-invasive, multimodal, non-opioid, neuromodulatory, patient-controlled, and empowering therapies to improve personal and population health.

In some embodiments, the present platform invention can be configured in single or combinations of contemplated micro and macro delivery systems for subjects, including mammals, in need of a multitude of currently known or unknown, but future artificial intelligence-determined, iterations and engineered therapies. An example of a micro device includes a device capable of strategic device electrode placement to provide stimulation or modulation of various nerves, including auricular vagal, with or without active transdermal compound delivery, or stimulation or modulation of other selected targeted nerves, including peripheral nerves, directly, or via incorporation into an epidural or intrathecal catheter. An example of a macro device may include a device of a selected size for targeting the spinal cord, dorsal root ganglia, dorsal horn, thalamus, midbrain, or other specific targets of the central nervous system.

In some embodiments, the present platform invention comprises a device design comprising a modular system of components with discrete functions. In some embodiments, modular components, including but not limited to sensors, may be positioned in or on the platform device, ventral to one or more electrodes, such that converse opposing electromagnetic vectoring magnetic fields produced by the one or more electrodes may be used to influence compounds, including but not limited to therapeutics, that may be stored, and/or incorporated in, said modular components, so the compounds may be passively, or active transdermal delivered to a target as influenced by the vectored magnetic moment of the converse opposing and vectoring magnetic fields.

In some embodiments, the present invention features functional systems that are self-contained onto a single patch, or onto more than one patch, that is/are either single-use or recharged, reconstituted, and/or refilled multiple-use.

In some embodiments, the present platform invention features a wearable, transcutaneous, non-invasive, multimodal, multi-mechanism of action (including, but not limited to, analgesic action), neuromodulatory device (which may in some embodiments be a single device) for active delivery of transdermal compounds, including analgesic compounds, to offset nociception whether synergistically, dependently, or independently of the device's multiple, multimodal, mechanisms of action, to a mammal or other subject in need of analgesia.

In some embodiments, the present platform invention features a wearable analgesic device comprising a reservoir, including but not limited to a hydrogel reservoir, with or without a fill port, for maintaining analgesic or therapeutic compounds for active transdermal delivery (i.e., via implied force) or passive transdermal delivery.

In some embodiments, the present invention features a wearable neuromodulatory device capable of generating and projecting inhomogeneous converse opposing dynamic rotational electromagnetic fields, pulsed electromagnetic fields, or vectored rotating dynamic electromagnetic field gradients in single or multiple pairs, or both, independently or dependently in series or parallel, thereby capable of assisting in acute transdermal drug delivery and drug vectoring in order to manage, treat, resolve, and/or eliminate pain in a subject (e.g., a mammal) in need of therapy.

In some embodiments, the present invention features a wearable multimodal analgesic platform device having one or more conductive surfaces to contact the skin for vectored delivery of electromagnetic energy to assist in active transdermal drug delivery and/or pain management.

In some embodiments, the present invention features a wearable platform device capable of ionoto-magnetic projected, vectored, electromagnetic active transdermal delivery, perpendicular and lateral to a target tissue, capable of “drawing or pushing”, or vectoring, therapeutic molecules into the epidermis, dermis, hypodermis or deeper subcutaneous or neural tissue targets and/or anchoring or “holding” the therapeutic compound(s) at the treatment target tissue, and/or capable of maintaining and vectoring the therapeutic compound(s) at the therapeutic target tissue, thereby sustaining or extending the duration of effectiveness, thereby increasing efficiency and efficacy, and decreasing potential systemic toxicity of the therapeutic in a subject (e.g., a mammal) in need of therapy.

In some embodiments, the present platform invention vectors compounds along a coronal plane, a sagittal plane, or a transverse plane, or some combination thereof. In some embodiments the present invention may alter the vector's magnitude along which compounds are vectored, thereby delivering compounds to different tissues that are either deeper or more superficial than others, depending on the vector's magnitude. In some embodiments, the present invention may precisely alter, or maintain, a therapeutic compound on target, dwell time, within a specific tissue. In some embodiments, a therapeutic compound dwell time may be vectored, modulated, by alternating between direct current polarities, or by using alternating current, or some combination thereof.

In some embodiments, the present platform invention features two, or more, electrodes of different sizes or configurations, allowing for the production and generation of different current densities and/or magnetic dipole moment between adjacent electrodes. In some embodiments, this configuration allows for the production and spatial prediction of the vectored conical electromagnetic field effect on a therapeutic compound. In some embodiments, the production, projection, vector of conical electromagnetic fields allows for the modulation of therapeutic compound concentration within a targeted tissue site. In some embodiments, this allows for vector “focusing” of a therapeutic compound, or, in other words, modulating the spatial concentration of therapeutic compounds in a precise single targeted tissue, or two or more precise but different target tissues. In some embodiments, compound concentration may be greater at the apex or vertex of the conical electromagnetic field than it is at the base of the conical electromagnetic field.

In some embodiments, the present invention uses one or more of the methodologies taught herein to provide for more efficient and precise active transdermal vector delivery of compounds through different types of anatomic features, of tissue and skin. For example, the thick skin of the soles of the feet and palms of the hands is characterized by the presence of the stratum lucidum within the epidermis. Penetration of certain skin features, such as the stratum corneum, stratum lucidum, or others, may require the use of different vector methodologies disclosed herein for the efficient transdermal delivery of compounds. For example, the present invention may be used for the active transdermal delivery of fluorouracil to treat a keloid, or the active transdermal delivery of an antineoplastic agent to treat skin cancers or biologic to treat autoimmune conditions.

In some embodiments, the present platform invention features light-emitting photobiologic diodes, and/or photobiomodulation lasers that promote vasodilation, collagen production (red light), antibacterial (blue light), or other wavelength, photobiomodulation analgesic, anti-inflammatory, or other physiological effects. In some embodiments, photobiomodulation may be defined as “a therapeutic technique that uses low-intensity light, typically in the red or near-infrared spectrum, to stimulate cellular processes and promote healing.”

In some embodiments, the present invention incorporates a ring design, protecting and maintaining the refillable reservoir.

In some embodiments, the present invention features a protective bumper and related structure that can be used as a medium to ultrasonically and/or kinetically move the therapeutic or drug molecule into the dermis, epidermis, hypodermis or other targeted sites for therapy to a subject (e.g., a mammal) in need thereof.

In some embodiments, the present platform invention incorporates a dynamic, or rotating converse opposing electromagnetic vectoring field for blocking nociceptive transduction and transmission, and/or features an active transdermal electromagnetic pulse vector generator projecting and modulating nociceptive signaling, somatosensory signaling, thereby enhancing analgesia pain perception. In some embodiments, the present invention also provides an active transdermal analgesic therapeutic compound delivery system designed to provide vector targeted analgesia to a subject (e.g., a mammal) in need of analgesia.

In some embodiments, the structure of the platform device of the present invention may be self-contained, autonomous, such that the device does not require a tethered control mechanism.

In some embodiments, the device of the present invention may be rechargeable in its standalone configuration using currently available non-rechargeable batteries, rechargeable lithium-ion batteries, and/or new carbon source capacitors like graphene.

In some embodiments the device of the present invention may have removable reservoirs to enable the drug to be pre-cartridge in form and thus simply attached to the dermal/mucosal electrode patch.

In some embodiments, the device of the present invention may contain secure remote-controlled features, via technologies such as Bluetooth™, and the like, to enable the user to control patch functions through their cellphone or another Bluetooth™-enabled device or device enabled with a similar communication technology. In some embodiments, the present invention features a Bluetooth™ or other wireless connection to another device (for example, a smartphone) running a software program interfacing with the device, thereby providing patient-facing opportunities for compliance, the reporting of patient reported outcomes, categorization of treatment parameters for diagnosis, and the like. In one non-limiting example, the present invention may incorporate a geolocation function that can measure one or more of the following parameters: a range of motion of a given joint, (for example, the knee), gait, fall risk, activity level, sleep cycle, timed “up and go” test, and the like.

In some embodiments, the device of the present invention may have a software app integrated into the user interface.

In some embodiments, the wearable device of the present invention may have indicator LEDs and/or alarms/notifications that provide to the user or practitioner real-time 1) reservoir volume, 2) skin and/or device temperature, conductance, and/or impedance 3) battery status, 4) skin pH, 5) SaO₂, 6) hemoglobin, 7) Hemoglobin a1c, 8) glucose, or other physiologic measurements, parameters, or biometric information to monitor device function, and/or provide real-time feedback, to improve therapeutic safety and efficacy in a subject (e.g., a mammal) in need of therapy.

In some embodiments, the device of the present invention may contain and support a memory function to report functional status and device use to report compliance and duty station time.

In some embodiments, the biometric computational parameters/aspects of the device of the present invention, in stand-alone form, or with a wireless or tethered or hard-wired controller, may have artificial intelligence (Al) functions to record and learn from patient usage based on skin type, vectored parameters, injury type, injury location, or pathology, for determining and implementing best evidence-based methods of drug and pain therapeutic delivery for future applications on new patients.

In some embodiments, the present invention features Al functions that will produce tabulated profiles of patient biographics, biometrics, and characteristics of treatment profiles, and/or protocol iterations, that best match optimal treatment parameters to individual patient need, thereby promoting best evidence-based performance, outcome, safety, and efficacy, and population health.

In some embodiments, the present invention is characterized by modularity of the platform and/or patch and/or device that may allow for other technologies, or modules/devices, to access and use the base adhesive or attachment to the skin for alternative treatment protocols—i.e., delivery of holistic compounds.

In some embodiments, the device of the present invention may also utilize, deliver, and vector holistic compounds and/or molecules that are prepared or modified to achieve optimal pharmacodynamic, pharmacokinetic, ionic, kinetic, photonic, or electromagnetic active transdermal delivery. In some embodiments, the present invention may utilize ionic force, iontophoretic force, iontophoresis, magnetophoretic force, similar force(s), or some combination thereof, to active transdermal deliver one or more compounds to a subject in need thereof. In some embodiments, the present invention may use ionic force, iontophoretic force, iontophoresis, magnetophoretic force, similar force(s), or some combination thereof to vector a therapeutic to a tissue target then “hold” or “sustain” the therapeutic in place, dwell, in a precise spatial tissue plane. Spatial dwell is enabled using reciprocal vector pattern frequencies, ionic force, iontophoretic force, iontophoresis, magnetophoretic force, or some combination thereof via use of different pattern frequencies. In some embodiments, this reduces the available therapeutic of the compound entering systemic circulation, thereby reducing the risk of systemic toxicity associated with said compounds.

In some embodiments, the present invention may utilize ionic force, iontophoretic force, iontophoresis, magnetophoretic force, similar force(s), or some combination thereof in a complementary or synergistic fashion. For example, iontophoretic force, while effective at providing for transdermal delivery, causes pain at the site at which iontophoretic force is applied. In some embodiments, the present invention provides for the application of magnetophoretic force or other electromagnetic stimulation or manipulation to anesthetize, or provide analgesia, to the target to which vectored iontophoretic force is applied, thereby reducing pain associated with the use of iontophoretic force, thereby increasing patient compliance and allowing for increase improved tolerability to vectored active transdermal delivery of compounds, including therapeutic compounds and other compounds of increased molecular weight. In some embodiments, this effect or similar effects may be described as “exponentially synergistic,” such that the combined advantageous effects achieved by the invention, including but not limited to those advantages obtained via the use of ionic force, iontophoretic force, iontophoresis, magnetophoretic force, similar force(s), or some combination thereof, with or without transdermal or transcutaneous delivery of chemical compounds (e.g., active pharmaceutical ingredients) is not simply additive of the individual beneficial effects of each individual treatment modality, nor simply synergistic (i.e., something more than additive), but instead increases exponentially depending on the amount of each treatment modality used.

In some embodiments, the present platform invention may utilize concomitant magnetic fields, electric fields, or both, to block nociception. The neurochemical reaction to tissue trauma, nociception, is known as transduction. In chronic pain, these nociceptive neuronal cells can become sensitized, which is sometimes caused by neurons becoming more permeable to sodium ions, which increases the cell's resting membrane potential, moving it closer to the cell's threshold potential. Therefore, in chronic pain, smaller changes in membrane potential can cause a nociceptive neuron to reach its threshold potential, because the state of chronic pain causes nociceptive neuron resting potential to be closer to its threshold potential, thereby causing the nociceptive neuron to reach its threshold potential more frequently and/or more easily, thus propagating an action potential and causing nociception and chronic pain. By applying a magnetic field (including, but not limited to, those produced by converse opposing electromagnetic vectored fields) to a neuron with higher resting membrane potential (e.g., a neuron in a patient with chronic pain), the magnetic converse opposing vectored field can alter the neuronal membrane's permeability to sodium, thereby bringing resting membrane potential back down to its healthy (i.e., not chronic-pain involved) resting membrane potential, thereby requiring a larger stimulus to reach its threshold membrane potential, thereby limiting the propagation of action potentials within nociceptors, thereby treating chronic pain.

In some embodiments, the present invention may utilize converse opposing electromagnetic vectoring fields to block nociception. In some embodiments, the present invention may utilize converse opposing electromagnetic vectored fields to block nociceptive signaling in A-delta or C-fibers allowing non-painful recruitment, transcutaneous stimulation and stimulus amplitude up-ramping of vector targeted afferent A-Beta neurons. In some embodiments, the present invention may be used to periodically increase or “ramp up,” or decrease or “ramp down” the vectored intensity, magnitude, frequency, pulse width, or strength of an electromagnetic or transdermal electric field produced by the present invention, thereby attenuating or blocking nociception, especially in unmyelinated fibers. In some embodiments, the present invention utilizes Al technologies to target interindividual differences in pain response. In some embodiments, the present invention utilizes Al technologies to measure or otherwise determine changes in therapies provided to enhance analgesic efficacy. In some embodiments, the present invention incorporates accelerometers, geo tracking sensors, or the like, thereby enabling the measurement and analysis of certain clinical biometric parameters, for example, gait, pace, range of motion, gait stability and the like. In some embodiments, the present invention may correlate the measurement of clinical objective parameters with certain treatment parameters, for example, in some embodiments the present invention may vector the transdermal delivery of a drug along a certain vector to a tissue target with a certain vectored magnitude to allow for an improved range of motion in a joint treated with the present invention. In some embodiments, the present invention features internet connectivity, wherein a patient, healthcare provider, or other person may report, surveil, or monitor patient-reported-outcomes or may monitor certain parameters measured and reported/transmitted by the present invention, including but not limited to biophysical measurements such as blood flow, blood glucose, blood pH, blood oxygen saturation, and the like. In some embodiments, the present invention accomplishes monitoring of certain parameters, including but not limited to biophysical measurements such as blood flow, blood glucose, blood pH, blood oxygen saturation, levels of Interleukin-6 or other interleukins or other inflammatory cytokines, and the like via the use of microneedles incorporated into the electrode and/or hydrogel patch. In some embodiments, the microneedles may also breach the stratum corneum thereby enhancing active transdermal delivery of a therapeutic. In some embodiments, measurement of these parameters may be used to show objective improvement of certain medical conditions, for example, osteoarthritis of the knee.

In some embodiments, the present invention may be used for the treatment, monitoring, surveillance, or a combination thereof, of conditions or disease states other than pain. For example, in some embodiments, the present invention may be used to measure blood glucose and transdermal deliver insulin in a manner responsive to simultaneous, real time, blood glucose measurements. In some embodiments, the present invention may be used to locally deliver drugs with considerable systemic toxicity, vectoring and increasing, decreasing, or maintaining, the time on target or dwell time, hereby reducing systemic toxicity, and increasing therapeutic efficacy to a patient in need thereof. For example, in some embodiments, the present invention may be used to deliver imiquimod, 5-fluorouracil, or other drug with considerable systemic toxicities vectored to a portion of the patient's skin in need of treatment thereof, maintaining time-on-target and/or dwell time, thereby reducing the patient's systemic exposure to these treatment modalities, thereby reducing systemic toxicity and enhancing therapeutic time on target, efficacy, and safety.

In some embodiments, the present invention may feature a single-use patch containing a single-use mechanical feature, such as a feature that imposes a one-time use of the device, or that prevents refilling of the device reservoir, or that electronically shuts-off after a preset number of duty cycles to disable the device.

In some embodiments, the present invention may feature a tethered controller that may have a number of preset usages, duty cycles that once exceeded temporarily or permanently disable the device.

In some embodiments, the device of the present invention may include real-time monitoring of the effects of the drug/compound that it is delivering, such that Al functionality or similar functionality may be used in controlling and metering the dose by patient activity cycles—for example, in delivering insulin to a diabetic patient.

In some embodiments, the device of the present invention may have the ability to singularly function as a monitoring device, for example, when awaiting a refill, to adjust and respond to reported pain levels, clinical, and/or therapeutic outcomes.

In some embodiments, the device of the present invention may control the inertial characteristics of the drug/compound molecule by an external vectoring platform component and/or element that controls the therapeutic penetration velocity, depth, and/or directional vector (including in consideration of vascular structures and tissue types), and/or dwell time at the vector targeted treatment site, thereby providing maximal therapeutic efficacy, efficiency, and safety.

In some embodiments, the device (which, in some embodiments, may be a platform device) of the present invention is designed with a notification system that actively alerts the wearer, or individual monitoring the system, such that said person is made aware of device performance characteristics, status, feedback, or suggested intervention, thereby promoting optimal therapeutic efficacy and safety.

In some embodiments, the present invention features Al functions to actively provide real time, simultaneous, therapeutic recommendations regarding the present and future use of the device's features and/or functions, thus tailoring it to individual usage patterns to achieve optimal treatment parameters, thereby improving patient and population health.

In some embodiments, the device of the present invention may have the ability to upload packets of data to a receiver, thereby allowing a practitioner or web-based monitor to monitor/surveil patient compliance and device performance, outcome metrics, efficacy, and safety to thereby improve patient and population health.

In some embodiments, the present invention may feature a drug/therapeutic that may be injected into a reservoir or be contained in a replaceable, patient-specific, or therapeutic-specific cartridge or hydrogel microneedle patch or other transdermal delivery system. In some embodiments, the present wearable device (which in some embodiments is a platform) is placed over a targeted tissue that has been injected or infiltrated with a therapeutic compound to influence or vector the spread of the therapeutic compound, or cease movement of the therapeutic compound, thus, increasing the therapeutic dwell on station duty cycle/time. For example, an analgesic therapeutic or compound may be injected, then vectored, or directed into or to a targeted tissue or fascial plane, increasing the time on target or dwell time of the analgesic, thereby maintaining therapeutic efficacy. Continuous vectored non-invasive active transdermal delivery to a targeted tissue, similar to an invasive percutaneous catheter infusion delivery system. In both effective therapeutic pharmacokinetic delivery systems, both examples delivering a therapeutic to a tissue target, the platform innovative device may vector the delivered therapeutic to the tissue target maximizing pharmacodynamic effect by vectoring the therapeutic, maximizing therapeutic time on target, dwell time.

In some embodiments, the present invention avoids/eliminates local discomfort or pain with increasing stimulation amplitude at or near to the target stimulation electrode placement that is commonly reported in the prior art. These prior art devices may also cause painful muscle stimulation and spasm/contraction that is avoided/eliminated using the present invention. The present invention provides for vectoring and active transdermal delivery of a variety of analgesic compounds, including sodium channel blockers, non-steroidal anti-inflammatory drugs, and NMDA receptor antagonists, as to the targeted tissue as a single delivery or a continuous delivery. Without wishing to limit the present invention to any theory or mechanism it is believed that magnetic converse opposing electromagnetic vectoring fields allow for higher amplitudes of direct current to be used to facilitate drug delivery, allowing for differentiation in speed, concentration, time to target, time on target, and provision of continued polarity switching of the direct current to maintain an “on-site” or stationary position of the compound at the site or action or injury.

In some embodiments, the device of the present invention may have a microthermal imaging capability to monitor vascular changes, edema, injury regression/progression, and/or treatment.

In some embodiments, the present invention features a device that may monitor heart rate, peripheral carbon dioxide tension, oxygen saturation, blood pressure, hemoglobin A1c, skin conductance, edema, or other signs of illness, discomfort or pain, and through Al functions, may self-determine the best protocol for maximum efficacy, efficiency, and safety of therapy.

In some embodiments, the device platform of the present invention may also feature an internal accelerometer and geolocation function, capable of monitoring motion of an area of the body, range of motion of a joint, or body motion as a whole, for measuring range of motion, activity, sleep cycle, gait, balance or fall risk, or specific outcome metrics like the “Timed Up and Go” test.

In some embodiments, the wearable platform device of the present invention may be incorporated into other fitments, braces, pads, castings, footwear, insoles, bandages, medical helmets, or other compatible housings or positioning applications, to maintain device positioning, targeting, and/or to improve comfort, compliance, and efficacy.

In some embodiments, the device of the present invention may, through Al, monitor heart rate and drug infusion rate to thereby optimize and change drug/compound delivery to patient vasculature, and/or or to otherwise alter other therapeutic mechanisms of action, to prevent diffusion, over disbursement, or overdosing of the drug or compound.

In some embodiments, the platform device of the present invention may function independently as a measurement/monitoring device for post-treatment measurement/monitoring of an injury or disease, and/or through Al, determine if the patient requires therapeutic titration or additional drug/compound treatments or other therapeutic interventions provided by the platform.

In some embodiments, the present invention may have a therapeutic hydrogel and/or an analgesic hydrogel to maximize efficiency and delivery of active transdermal therapies and/or pain management protocols.

In some embodiments, the present invention may be designed to be used for cranial applications, craniofacial/dental applications, and/or for applications for treatment of specific dermatologic conditions and for use in other specific device locations, for the pharmacologic treatment of tumors or skin disorders, and/or for direct cortical tissue applications such as treatment of Parkinson's disease, Multiple Sclerosis, epilepsy, or other selected oncologic, cardiovascular, endocrine, neurological, or dermatologic conditions.

In some embodiments, the present invention may use power storage technologies such as lithium-ion battery technology, solid-state battery technology, graphene power storage, or other high-capacitance storage technologies to improve and prolong duty cycle or monitoring.

In some embodiments, the present platform invention may feature a wearable device wherein the vectored frequency, pulse width, pulse depth, pulse waveform, pulse rate, and pulse amplitude or continuous stimulation delivered may be internally-controlled or ramped, including the delivery of low, high and ultra-high frequency transcutaneous electrical neuromodulation, or vectored cycles thereof, to targeted tissue, vectoring, photonic, iontophoretic, and/or magnetophoretic therapy of active pharmaceutical ingredients, allopathic compounds, holistic compounds, biological substances, chemicals, and/or any combination thereof. In some embodiments, the present invention may independently utilize ultra-high frequency stimulation to vector neuromodulation and/or analgesia. In some embodiments, the present invention may utilize transcutaneous electrical nerve stimulation (TENS) to vector, induce nociceptive neuromodulation, anesthesia, and/or analgesia. In some embodiments, the present invention may target specific peripheral nerves, including but not limited to cranial nerves or the auricular vagus nerve, as well as the somatosensory and/or central nervous system. In some embodiments, the present invention may vector and/or induce neuromodulation and/or analgesia or anesthesia, via somatosensory and central neuronal pacing, e.g., recruiting and pacing A-beta neurons. In some embodiments, the present invention may provide multimodal analgesia, for example, by combining one or more of the aforementioned modalities with active transdermal delivery and vectoring of a compound, e.g., one or more active pharmaceutical ingredients. In some embodiments, the active pharmaceutical ingredients delivered by the present invention may include bupivacaine, ketorolac, or ketamine, or various combinations thereof, including bupivacaine, ketorolac, ketamine, a TRPV agonist, a central alpha two agonist, or any of the active pharmaceutical ingredients or other compounds or components referenced in U.S. Pat. Nos. 10,098,872B1, 11,559,521B2, 11,992,484B2, 11,992,485B2, U.S. Ser. No. 18/069,006, U.S. Ser. No. 18/543,781, and/or U.S. Ser. No. 18/543,991, the specification(s) of which is/are incorporated herein in their entirety by reference. In some embodiments, the active pharmaceutical ingredients vectored and/or delivered by the present invention may include a combination of active pharmaceutical ingredients comprising bupivacaine, ketorolac, and ketamine. In some embodiments, the active pharmaceutical ingredients vectored and/or delivered by the present invention may include a local anesthetic, an anti-inflammatory (for example, a non-steroidal anti-inflammatory drug, i.e., an NSAID), or an NMDA receptor antagonist, or various combinations thereof, including a local anesthetic, an anti-inflammatory (for example, a non-steroidal anti-inflammatory drug, i.e., an NSAID), and an NMDA receptor antagonist.

In some embodiments, the device of the present invention (which in some embodiments may be a platform) may also be used on animals in a veterinary environment.

In some embodiments, the present invention (which in some embodiments may be a platform) features one or more electrode current, ionic, and/or magnetophoretic dispersion plates that may be perforated with at least one hole, several holes or apertures, and/or permanent, biodegradable, or dissolving microneedles.

In some embodiments, the present invention features current dispersion plates that may be in direct or indirect contact with the vectored transdermally delivered material.

In some embodiments, the present invention features a device (which in some embodiments may be a platform) that may also contain elements of graphene, which provides for enhanced or differential function ability.

In some embodiments, the present invention features a device having conductive surfaces contacting the patient's skin, allowing measurement of EMG (electromyography) signals and/or selective skin conductance. In some embodiments, the present invention comprises a skin-contacting component, or components, further comprising an electrode capable of measuring an EMG signal via the EMG electrode. In some embodiments, the electrodes of the present invention are multimodal and are capable of multiple functions, including but not limited to measuring EMG signals, and/or any of the other functionalities disclosed herein.

In some embodiments, the present invention features a device (which in some embodiments may be a platform) designed and modified to deliver a sodium channel blocker, NMDA receptor antagonist, cyclooxygenase inhibitor, alpha agonist, TRPV agonist, or other analgesic, anti-inflammatory, or other active pharmaceutical ingredient, other allopathic or homeopathic compound, wherein said compound may be actively, delivered via transdermal delivery, and/or vectored to a tissue target.

In some embodiments, the present invention features a device (which in some embodiments may be a platform) having transdermal componentry, including but not limited to materials such as hydrogel, adhesives, microneedles, and the like, to form adhesion and/or enable molecular material transfer and/or vectoring from the device onto and into the skin and other tissue of a subject in need thereof.

In some embodiments, the present invention features a control unit (which in some embodiments may be a platform control unit). In some embodiments, the control unit is a printed circuit board. In some embodiments, the control unit incorporates a microprocessor. In some embodiments, the control unit is a printed circuit board with an integrated power source. In some embodiments the platform is wirelessly connected to a smartphone, tablet, watch, or other acceptable device allowing integrated connectivity for biometric measurement, and/or Al control and coordination of complex vectoring commands

In some embodiments, the present invention (which in some embodiments may be a platform) comprises, a wearable, multimodal device comprising: a power source; a control unit; at least one electrode or therapeutic API adhesive hydrogel capable of generating and projecting electric and magnetic fields, a therapeutic reservoir containing at least one chemical compound; a skin-contacting component; and fitment to house the remainder of the components wherein the power source is electrically connected to both the control unit and the electrode, wherein the control unit is functionally connected to the therapeutic reservoir, wherein the therapeutic reservoir is fluidically coupled to the skin-contacting component, wherein the control unit is configured to control the delivery of electrical power from the power source to the electrode to thereby generate and vector electric and magnetic fields, wherein the control unit is configured to control the vectored delivery of chemical compound from the therapeutic reservoir to the skin-contacting component, wherein the electric and magnetic fields generated by the electrode are projected at least partially into the epidermis of a subject (e.g., a mammal) in need of analgesia, and wherein the skin-contacting component delivers the chemical compound to at least the epidermis of the subject (e.g., a mammal) in need of analgesia.

### EMBODIMENTS

The following embodiments are intended to be illustrative only and not to be limiting in any way.

In an exemplary embodiment, the present invention uses a dynamic rotational converse opposing ferromagnetic, or electromagnetic vectoring field, a pulsed electromagnetic, and/or an electromagnetic oscillating or rotational magnetic field to block nociceptive transduction and allow for enhanced transcutaneous transdermal electrical nerve stimulation. In some embodiments, the present invention projects rotational dynamic converse opposing vectoring magnetic fields to induce analgesia in a subject in need thereof. The exemplary embodiment produces, projects, vectors, and enhances analgesia by modulating and/or neuromodulating nociceptive transduction and nociceptive transmission and signaling. Furthermore, an active transdermal, active pharmaceutical ingredient delivery system is combined with, or incorporated into, the design of the multimodal neuromodulatory pain management platform to deliver, vector, and sustain an analgesic compound or other therapeutic compounds to targeted tissues and targeted somatosensory and/or cortical regions of the mammalian anatomy.

Embodiment 1: A wearable multimodal device comprising: a skin-contacting component; a therapeutic reservoir containing at least one chemical compound therein, wherein the therapeutic reservoir is fluidically coupled to the skin-contacting component; at least one electromagnetic field generator capable of generating and projecting electric and magnetic fields; a power source electrically connected to the at least one electromagnetic field generator; a control unit electrically and/or electronically and/or wirelessly connected to the power source and functionally connected to the therapeutic reservoir; wherein the control unit is configured to control the delivery of electrical power from the power source to the electromagnetic field generator thereby generating electric and magnetic fields; wherein the control unit is configured to control the delivery of the at least one chemical compound from the therapeutic reservoir to the skin-contacting component; wherein the electric and magnetic fields generated and projected from the electromagnetic field generator are projected at least partially into an epidermis of a subject (e.g., a mammal) in need of therapy or analgesia, and wherein the skin-contacting component delivers the chemical compound to at least the epidermis of the subject (e.g., a mammal) in need of therapy or analgesia.

Embodiment 2: The wearable, multimodal device of embodiment 1, wherein the device delivers the, at least one, chemical compound via, at least one, of active transdermal delivery or passive transdermal delivery.

Embodiment 3: The wearable, multimodal device of any one of embodiments 1-2, wherein the, at least one, electromagnetic field generator is, at least one, of the following: at least one electrode or an array of converse opposing electrodes.

Embodiment 4: The wearable, multimodal device of any one of embodiments 1-3, wherein the, at least one, electromagnetic field generator generates and projects electric and magnetic vectored fields in at least one of the following modalities: transdermal or transcutaneous, at least partially into an epidermis of a subject (e.g., a mammal) in need of therapy or analgesia.

Embodiment 5: The wearable, multimodal device of any one of embodiments 1-4, wherein the skin-contacting component further comprises one or more of the following: an electrode, an electrically conductive material, an electromagnetic field generator, or an electrically conductive hydrogel, wherein said hydrogel contains one or more transdermal delivered chemical compound.

Embodiment 6: The wearable, multimodal device of any one of embodiments 1-5, further comprising at least one modular component, wherein the, at least one, modular component performs a therapeutic or diagnostic function and wherein the, at least one, modular component is modularly replaceable with another at least one, other modular component.

Embodiment 7: The wearable, multimodal device of any one of embodiments 1-6 further comprising one or more of the following additional components: a transcutaneous electrical nerve stimulation (TENS) unit, an interferential waveform unit, a high-frequency electrical stimulation unit, an ultra-high frequency electrical stimulation unit, or a microcurrent stimulation unit.

Embodiment 8: The wearable, multimodal device of any one of embodiments 1-7 further comprising at least one sensor operatively connected to the control unit and configured to deliver at least one feedback signal to the control unit, wherein the control unit responds to the at least one feedback signal by altering at least one modifiable parameter of the wearable, multimodal device.

Embodiment 9: The wearable, multimodal device of any one of embodiments 1-8, wherein the at least one sensor detects, at least one, of reservoir volume, skin temperature, device temperature, skin conductance, skin impedance, battery status, skin pH, oxygen saturation, hemoglobin concentration, hemoglobin A1c, advanced glycation end products, glucose concentration, heart rate, drug perfusion parameters, drug infusion parameters, signs of inflammation, or signs of edema.

Embodiment 10: The wearable, multimodal device of any one of embodiments 1-9, wherein at least one sensor detects, at least one, of a pharmacodynamic or pharmacokinetic parameter of at least one chemical compound delivered by the multimodal device.

Embodiment 11: The wearable, multimodal device of any one of embodiments 1-10, wherein the electric and magnetic fields generated and projected by the at least one electromagnetic field generator comprises one or more of: converse opposing electromagnetic vectoring fields or dynamic rotating magnetic field gradients.

Embodiment 12: The wearable, multimodal device of any one of embodiments 1-11, wherein the electric and magnetic fields generated by the, at least one, electromagnetic field generator are generated and projected in at least one of single or multiple pairs.

Embodiment 13: The wearable, multimodal platform device of any one of embodiments 1-12, wherein the at least one electromagnetic field generator projects at least one of: ionoto-magnetic projected, vectored, or electromagnetic active transdermal delivery signals, perpendicular and lateral to a target tissue, thereby vectoring the at least one chemical compound into, at least one, of the epidermis, dermis, hypodermis or deeper subcutaneous, fascial, or neural tissue targets of a subject in need thereof.

Embodiment 14: The wearable, multimodal device of any one of embodiments 1-13, wherein the at least one electromagnetic field generator projects at least one of a converse opposing ferromagnetic or electromagnetic vectoring field for impeding nociceptive transduction and transmission, or a transdermal transcutaneous electromagnetic pulse that modulates nociceptive signaling, thereby enhancing analgesic signaling.

Embodiment 15: The wearable, multimodal device of any one of embodiments 1-14, wherein the device modulates at least one of the following parameters of the converse opposing vectoring ferromagnetic or electromagnetic field or transdermal electromagnetic pulse: frequency, width, depth, waveform, rate, or amplitude of stimulation.

Embodiment 16: The wearable, multimodal device of any one of embodiments 1-15, wherein the device controls at least one of the following characteristics of the chemical compound delivered by the device: inertial characteristics, penetration, velocity, depth, directional vector, dipole moment, charge density, polarity, size, or dwell time at a treatment site.

Embodiment 17: The wearable, multimodal device of any one of embodiments 1-16 further comprising at least one of an accelerometer and geolocation sensor, measuring at least one of the following: motion of an area of the body, range of motion of a joint, body motion as a whole, activity level, sleep cycle, clinical test metric, or clinical outcome. Embodiment 18: The wearable, multimodal device of any one of embodiments 1-17 further comprising a conductive surface for measuring at least one of electromyography signals or selective skin conductance of a subject wearing the device.

Embodiment 19: The wearable, multimodal device of any one of embodiments 1-18, wherein the at least one chemical compound comprises one or more of the following: a sodium channel blocker, NMDA receptor antagonist, cyclooxygenase inhibitor, alpha agonist, TRPV agonist, or other analgesic, anti-inflammatory, or another active pharmaceutical ingredient.

Embodiment 20: The wearable, multimodal device of any one of embodiments 1-19 further comprising a transdermal stimulating componentry comprising at least one of hydrogel, adhesives, or microneedles.

Embodiment 21: A method for administering drugs through a dermal or mucosal layer comprising the following steps: providing a wearable, multimodal device with at least one electromagnetic field generator capable of generating electric and magnetic fields to provide an effective magnetic sphere of influence at a desired site of action; providing a tissue permeable formulation comprising at least one chemical compound, which is positioned on the site of action; and positioning the wearable, multimodal device adjacent to said tissue permeable formulation wherein the magnetic sphere of influence of the magnetic field moves the chemical compound though the dermal layer by interaction with the magnetic moment of the chemical compound, sufficient to increase the movement of the chemical compound beyond the movement of diffusion alone.

Embodiment 22: The method of embodiment 20, wherein the wearable, multimodal device further provides at least one of a magnetic or electric field for the relief of pain.

Embodiment 23: The wearable, multimodal device of any one of embodiments 1 or 6, or embodiments 2-5 or 7-19, wherein the device comprises a platform device further comprising at least one modular component, wherein the, at least one, modular component performs a therapeutic or diagnostic function and wherein the, at least one, modular component is modularly replaceable with another, at least one, other modular component, thereby allowing the platform device to be modularly adjustable to perform at least one additional therapeutic or diagnostic function.

As used herein, the term “about” refers to plus or minus 10% of the referenced number.

Although there has been shown and described the preferred embodiment of the present invention (which in some embodiments may be a platform), it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. A wearable multimodal device (001) comprising:
a) a skin-contacting component;
b) a therapeutic reservoir (500) containing at least one chemical compound therein, wherein the therapeutic reservoir (500) is fluidically coupled to the skin-contacting component;
c) at least one electromagnetic field generator (200) capable of generating and projecting electric and magnetic fields;
d) a power source (600) electrically connected to the at least one electromagnetic field generator (200); and
e) a control unit electrically or wirelessly connected to the power source (600) and functionally connected to the therapeutic reservoir (500); wherein the control unit is configured to control the delivery of electrical power from the power source (600) to the electromagnetic field generator (200) thereby generating electric and magnetic fields; wherein the control unit is configured to control the delivery of the at least one chemical compound from the therapeutic reservoir (500) to the skin-contacting component;
wherein the electric and magnetic fields generated and projected from the electromagnetic field generator (200) are projected at least partially into an epidermis of a subject in need of therapy or analgesia, the skin-contacting component delivers the chemical compound to at least the epidermis of the subject in need of therapy or analgesia, and wherein the electric and magnetic fields generated and projected from the electromagnetic field generator (200) at least partially hold the at least one chemical compound in, on, or at a treatment site for a dwell time, wherein the treatment site comprises a site where the at least one chemical compound exerts at least one mechanism of action.

2. The wearable, multimodal device (001) of claim 1, wherein the device (001) delivers the, at least one, chemical compound either by active transdermal delivery, passive transdermal delivery, or both active and passive transdermal delivery.

3. The wearable, multimodal device (001) of claim 1, wherein the, at least one, electromagnetic field generator (200) is at least one of the following: at least one electrode or an array of converse opposing electrodes.

4. The wearable, multimodal device (001) of claim 1, wherein the, at least one, electromagnetic field generator (200) generates and projects electric and magnetic vectored fields in at least one of the following modalities: transdermal or transcutaneous, at least partially into an epidermis of the subject in need of therapy or analgesia.

5. The wearable, multimodal device (001) of claim 1, wherein the skin-contacting component further comprises one or more of the following: adhesives, microneedles, microtubules, an electrode, an electrically conductive material, an electromagnetic field generator (200), or an electrically conductive hydrogel, wherein said hydrogel contains one or more transdermal delivered chemical compound.

6. The wearable, multimodal device (001) of claim 1, wherein the wearable, multimodal device (001) is a platform device, further comprising at least one modular component, wherein the, at least one, modular component performs a therapeutic or diagnostic function and wherein the, at least one, modular component is modularly replaceable with another, at least one, other modular component, thereby allowing the platform device to be modularly adjustable to perform at least one additional therapeutic or diagnostic function.

7. The wearable, multimodal device (001) of claim 1 further comprising one or more of a transcutaneous electrical nerve stimulation (TENS) unit, an interferential waveform unit, a high-frequency electrical stimulation unit, an ultra-high frequency electrical stimulation unit, or a microcurrent stimulation unit.

8. The wearable, multimodal device (001) of claim 1 further comprising at least one sensor operatively connected to the control unit and configured to deliver at least one feedback signal to the control unit, wherein the control unit responds to the, at least one, feedback signal by altering at least one modifiable parameter of the wearable, multimodal device.

9. The wearable, multimodal device (001) of claim 8, wherein the, at least one, sensor detects at least one of reservoir volume, skin temperature, device temperature, skin conductance, skin impedance, battery status, skin pH, oxygen saturation, hemoglobin concentration, hemoglobin A1c, advanced glycation end products, glucose concentration, heart rate, drug perfusion parameters, drug infusion parameters, signs of inflammation, signs of edema, or a pharmacodynamic or pharmacokinetic parameter of at least one chemical compound delivered by the multimodal device.

10. The wearable, multimodal device (001) of claim 1, wherein the electric and magnetic fields generated and projected by the at least one electromagnetic field generator (200) comprises either converse opposing electromagnetic vectoring fields, dynamic rotating magnetic field gradients, or both.

11. The wearable, multimodal device (001) of claim 1, wherein the electric and magnetic fields generated by the, at least one, electromagnetic field generator (200) are generated and projected in either single and/or multiple pairs.

12. The wearable, multimodal platform device (001) of claim 1, wherein the at least one electromagnetic field generator (200) projects at least one of: ionoto-magnetic projected, vectored, or electromagnetic active transdermal delivery signals, perpendicular and lateral to a target tissue, thereby vectoring the at least one chemical compound into at least one of the epidermis, dermis, hypodermis or deeper subcutaneous, fascial, or neural tissue targets of a subject in need thereof.

13. The wearable, multimodal device (001) of claim 1, wherein the, at least one, electromagnetic field generator (200) projects, at least one, of a converse opposing ferromagnetic or electromagnetic vectoring field for impeding nociceptive transduction and transmission, or a transdermal transcutaneous electromagnetic pulse that modulates nociceptive signaling, thereby enhancing analgesic signaling.

14. The wearable, multimodal device (001) of claim 13, wherein the device modulates at least one of the following parameters of the converse opposing vectoring ferromagnetic or electromagnetic field or transdermal electromagnetic pulse: frequency, width, depth, waveform, rate, or amplitude of stimulation.

15. The wearable, multimodal device (001) of claim 1, wherein the device controls at least one of the following characteristics of the chemical compound delivered by the device: inertial characteristics, penetration, velocity, depth, directional vector, dipole moment, charge density, polarity, size, or dwell time at a treatment site.

16. The wearable, multimodal device (001) of claim 1 further comprising at least one of an accelerometer or geolocation sensor, and measures at least one of the following: motion of an area of the body, range of motion of a joint, body motion as a whole, activity level, sleep cycle, clinical test metric, or clinical outcome.

17. The wearable, multimodal device (001) of claim 1 further comprising a conductive surface for measuring at least one of electromyography signals or selective conductance of a subject wearing the device.

18. The wearable, multimodal device (001) of claim 1, wherein the, at least one, chemical compound comprises one or more of the following: a sodium channel blocker, NMDA receptor antagonist, cyclooxygenase inhibitor, alpha agonist, TRPV agonist, or other analgesic, anti-inflammatory, or another active pharmaceutical ingredient.

19. A method for administering drugs through a dermal or mucosal layer comprising the following steps:
providing a wearable, multimodal device (001) with at least one electromagnetic field generator (200) capable of generating electric and magnetic fields to provide an effective magnetic sphere of influence at a desired site of action; providing a tissue permeable formulation comprising at least one chemical compound, which is positioned on the site of action;
providing a tissue permeable formulation comprising at least one chemical compound, which is positioned on the site of action; and
positioning the wearable, multimodal device (001) adjacent to said tissue permeable formulation wherein the magnetic sphere of influence of the magnetic field moves the chemical compound though the dermal layer by interaction with the magnetic moment of the chemical compound, sufficient to increase the movement of the chemical compound beyond the movement of diffusion alone.

20. The method of claim 19, wherein the wearable, multimodal device (001) further provides at least one of a magnetic or electric field for the relief of pain.
